(19) United States
(12) Patent Application Publication
(10) Pub. No.: US 2003/0055388 A1
(43) Pub. Date: Mar. 20, 2003

(54) Urinator belt

(76) Inventors: Gary Clinton, Amityville, NY (US); Terry Clinton, Amityville, NY (US)

Correspondence Address: GARY CLINTON, 490 ALBANY AVE., AMITYVILLE, NY, 11701, US

(21) Appl. No.: 09/951,919

(22) Filed: Sep. 14, 2001

(51) Int. Cl. A61F 5/44; A61M 1/00

(52) U.S. Cl. 604/353; 604/540; 604/350

(57) ABSTRACT

What is disclosed is a non-invasive urine collecting device and method for storing urine therein. The device provides sanitary and modest means for concealing a catheter-type device underneath a man's clothing so that the tube cannot be observed, but the individual wearing said device may discharge urine into the tube which is stored therein until later removal. The device comprises a jock strap type harness with a pouch in front for securing the male penis into a nozzle which is fluidly coupled with a collection tube that extends downwardly towards a person's ankle and can be strapped to the individual's leg and clamped closed so as to collect the bodily fluids for disposal later.

### FIELD OF THE INVENTION

The present invention relates to medical devices for assisting incontinent male patients, and more particularly to male patients who lack bladder control and require a device which collects ur9ne discharges from the penis and contains the liquid urine until such time as an opportunity arises for the individual or caretaker to drain the collected fluid.

### BACKGROUND OF THE INVENTION

Individuals who suffer from weak bladders or who cannot independently utilize a toilet, such as handicapped or disabled individuals, frequently encounter embarrassing accidents as a result of their infirmities. Various devices have been developed which can collect urine and store it until a convenient opportunity presents itself to drain the urine in a sanitary manner. One typical example of such a device is a catheter, which requires the insertion of a tube into the male penis for introduction into the bladder. Such devices are intrusive, frequently painful and on many occasions can increase the risk of infection due to contaminants which adhere to the tubing which is inserted into the body.

U.S. Pat. No. 4,957,487 issued in the name of Gerow, discloses an external male urinary catheter and collection system which utilizes a sheath-like member for accenting the male penis, a negative pressure evacuation device community with the sheath-like member, the negative pressure causing the penis to elongate in a simulated direction and a thin-walled sealing portion of the sheath-like member to provide an air-tight seal about the elongated penis.

U.S. Pat. No. 4,581,026 issued in the name of Schneider discloses an external catheter and the methods of making and using such catheter for a male urinary incontinence collection system. The system also includes an adhesive attachment for holding the catheter in place. Catheter takes the form of a tubular sheath thin elastic material having a cylindrical body section, tapered neck section extending from the body section and a reduced drainage tube section projecting from the neck section. Within the sheath and constituting unitary part thereof is tubular inner sleeve of soft elastic material having a distal end portion disposed within the neck section and a proximal end portion merging with the sheath cylindrical body section. The sleeve tapers distally to define an annular space thereabout, and terminates in a reduced distal opening located in the immediate point within the next section.

U.S. Pat. No. 4,388,923 issued in the name of Heimreid discloses an external urinary drainage device. The drainage means is in the form of a condom which at the outer end terminates and/or is connected to a length of hose for carrying off urine. Condom is formed as a short condom which covers only the head (glans) of the penis. Extending from the outer end of the condom or from a length of hose is a sheathing body which when the condom surrounds the head of the penis and the foreskin has been drawn forward over the condom, can be made to encase the condom and foreskin, thereby providing a labyrinth seal.

### SUMMARY OF THE INVENTION

What is disclosed is a non-invasive urine collecting device and method thereof comprising a waist encompassing harness. The harness is equipped with a waist-strap, a pair of leg straps, and a pouch for retention of a male penis therein. A hose adaptor is coupled in fluid communication with said pouch. A tube for collecting urine is in turn coupled in fluid communication with the hose adaptor. A discharge orifice with means for clamping said discharge orifice allows the user to empty the retained urine at a convenient time.

In an alternate embodiment, the urine collecting device and method thereof is also provided with means such as a flexible strap, for securing the discharge orifice at the adjacent the discharge end to an individual's leg above the ankle, so as to avoid visual detection of said collection tube.

It is an object of the present invention to provide a sanitary, convenient and non-invasive means for incontinent male individuals to urinate into a containment tube which stores urine for later drainage.

It is yet another object of the present invention to provide an exterior catheter-type device which does not require intrusive introduction of foreign substances in the body which, in turn, increases risk of internal infection. It is yet another object of the present invention is to provide a long thin rubber tube that runs down inside of a user's pants leg attached to a jock strap-like band around the waist to secure a cup and tube in place.

It is yet another object of the present invention to provide an optional filter attached to the bottom of a clamp to eliminate odors.

It is yet a further object of the present invention to provide a conveniently installed jock strap type harness about the individual's waist and provides a rubber cup disposed over the penis for recoverable attachment thereto.

Another object of the present invention to provide a device which allows the user to comfortably urinate without embarrassment and associated mess.

Further object of the present invention to enable handicapped or disabled persons or persons with weak bladders to maintain their dignity, privacy without the need to ask for assistance in using a toilet and the embarrassment of wet clothing.

### DESCRIPTION OF THE DRAWINGS

FIG. 1 is an isometric view of the invention showing a fragmentary view of the tube.

FIG. 2 is a front tube of a male individual employing the device of the present invention;

FIG. 3 is a partial view of the end of the tube showing the clamp and a partial sectional view of the inner linings of the tube;

FIG. 4 is an illustration of the end of the tube and clamp, with the clamp removed, and urine flowing out the discharge end of the tube.

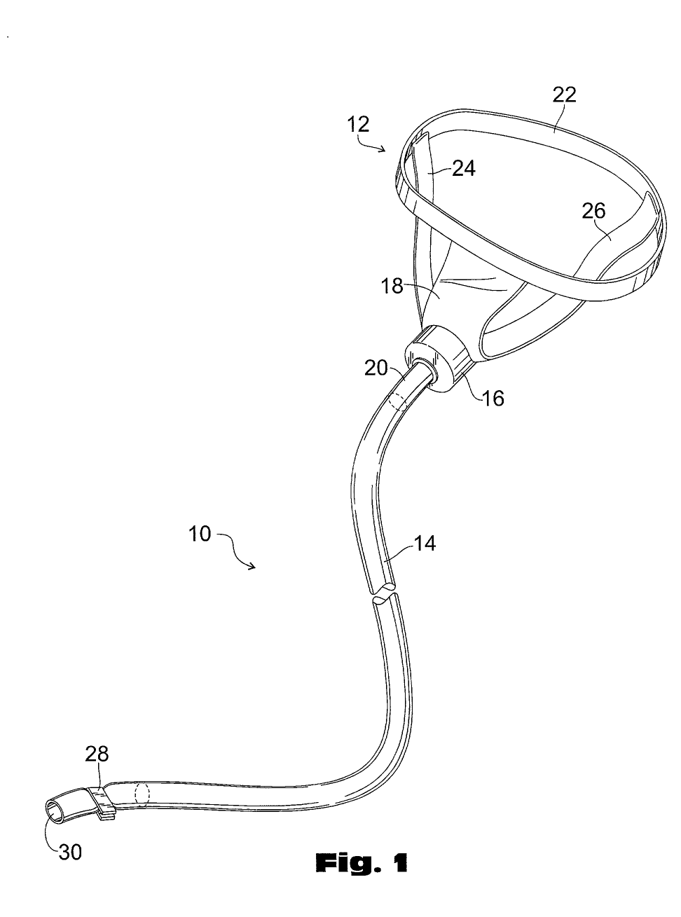

### DETAILED DESCRIPTION OF THE INVENTION

Referring first to FIG. 10, the urinator belt is generally designated as 10. A jock strap type device 12 is shown with a pair of straps 24, 26 connected in a triangular fashion. A front panel 18 which is intended to cover the male penis. A front panel 18 has an opening (not shown) which is fluidly coupled to nozzle 16 which has a discharge connector tube 20 which is coupled at the discharge end of nozzle 16 to tube collector 14. Tube collector 14 comprises a length of the hose sufficient to carry a quantity of urine equivalent to at least a normal bladder discharge. At the end of tube 14 is aperture 30. Clamp 28 is a spring retained clamp. When placed adjacent aperture 30 at the discharge end of collector tube 14, the urine is retained within collector tube 14 until such time as the user has an opportunity to drain the hose in a private and sanitary fashion.

The jock strap 12 is constructed as a typical jock strap wherein the elastic straps 24 and 26 attach one end to elastic waistband 22 which is intend to encompass the individual's waist and be elastically retained thereabout. Also attached to the waistband 22 is the front panel 18 which is generally a wider strip of material than the elastic bands 24, 26. The end of the panel opposite that connected at the waistband, is connected to the opposite ends of elastic straps 24, 26 at a three point intersection. This three point intersection and the attachments of the waistbands form leg openings for pulling the elastic waistband 22 over and between the legs and up around the individual's waist.

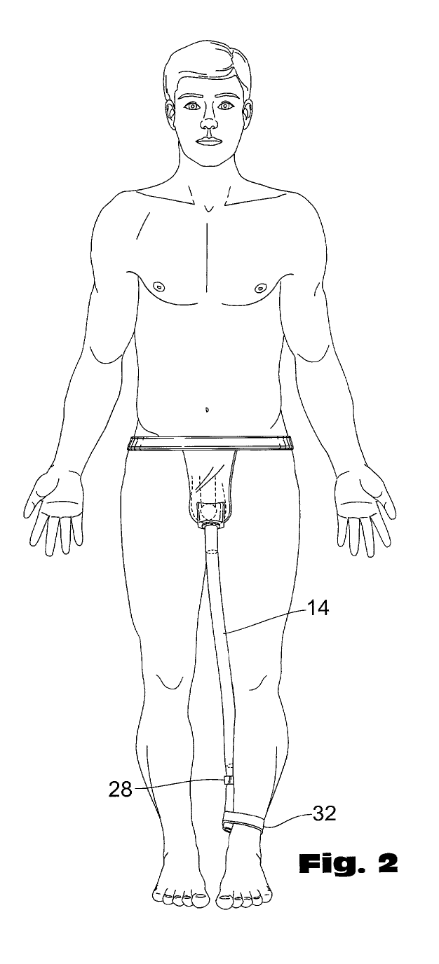

FIG. 2 illustrates a man in a front view showing the waistband fixed about the waist above the penis. The front panel 18 is shown with the genitalia shown in shadow behind the panel, and coupling the end of the penis into the nozzle 16 and therethrough to the collector tube 14 which is in turn strapped about the individual's ankle to the leg just above the ankle by ankle strap 32. The clamp 28 ensures a positive closure of the collector tube 14 so as to prevent the liquid from escaping.

Referring next to FIG. 3, the cutaway section shows outer wall 42 interior ring is inserted into the adjacent discharge end. The ring portion provides an inner wall 44 and a reinforcing mesh 46 which prevent the tube from collapsing adjacent the discharge aperture. The tube is clamped by means of clamp 28 adjacent the aperture 30 at the end opposite nozzle connection. The fragmentary sectional view in FIG. 3 indicates an illustration of the outer wall 42 and an inner wall 44 which provides a reinforcement. A reinforcing mesh is

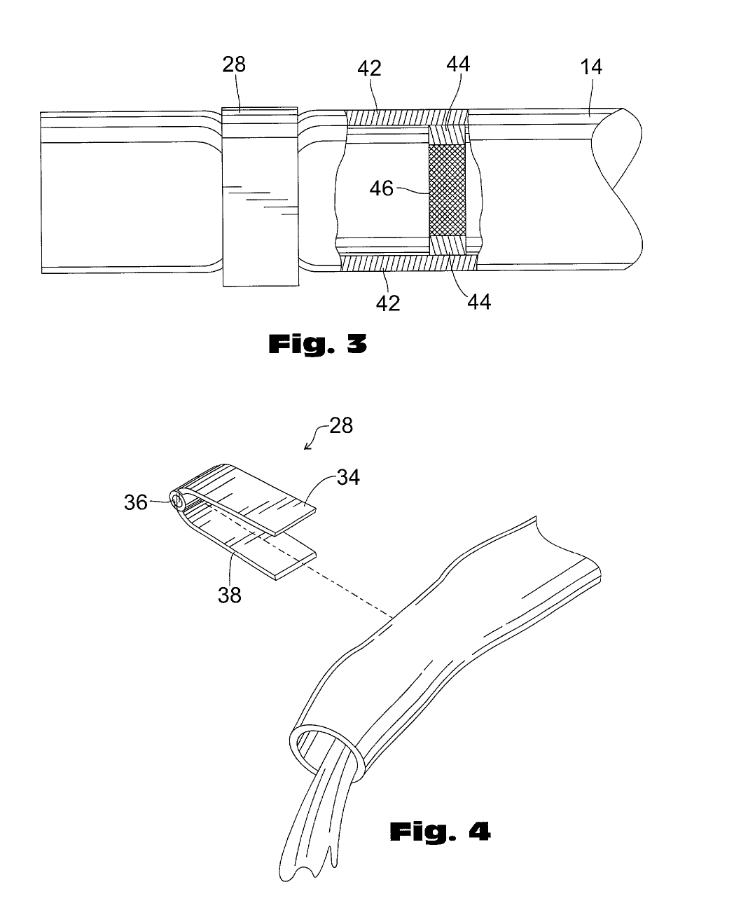

Referring next to FIG. 4, the clamp 28 is shown after removal from the collector tube 14. Liquid, presumably urine, is shown escaping from the discharge aperture 30. A pair of plates 34, 38 have connecting spring 36 at one end and are freely extended at the opposing distal ends. A spring portion holds the opposing plates normally in a compressed relation to each other to forcibly engage the flexible tube therebetween.

Also disclosed is a method of preventing accidental urine discharge, by means of: providing a waist harness with means for securing a male penis; connecting means for securing to a nozzle end; fluidly coupling said nozzle end to a tubular storage collector; providing a clamping means adjacent opposite end of said tubular storage means; and fastening a discharge end of said tubular means to an individual's leg adjacent and above the ankle, thereby concealing said tubular storage means within a man's pant leg so as to avoid visual detection.

According to the provisions of the patent statutes, we have explained the principal, preferred construction and mode of operation of my invention and have illustrated and described what we now consider to represent its best embodiments. However, it should be understood within the scope of the appended claims, the invention may be practiced otherwise than as specifically illustrated and described.

We claim:

1. A urine collecting device and method thereof comprising:
A waist encompassing harness, said harness having: a waist-strap, a pair of leg straps, and a pouch for retention of a male penis therein;
A hose adaptor in fluid communication with said pouch;
A tube for collecting urine, in fluid communication with said hose adaptor;
A discharge orifice;
and a means for clamping said discharge orifice.

2. The urine collecting device and method thereof is set forth in claim 1, wherein, also providing a means for securing the discharge orifice at the adjacent the discharge end, to an individual's leg above the ankle, so as to avoid visual detection of said collection tube.

3. The urine collecting device and method thereof as set forth in claim 1, wherein said hose adaptor is comprised of a tubular flexible sheath, having first and second ends with a sealed connection between said tube and said sheath first end, and a second sealed connection at said second end and said pouch for retaining penis.

4. The urine collecting device and method thereof as set forth in claim 1, wherein said tube for collecting urine is comprised of flexible surgical tubing.

5. The urine collecting device and method thereof as set forth in claim 1, wherein said tube for collecting urine is comprised of rubber tubing.

6. The urine collecting device and method thereof as set forth in claim 1, wherein said tube for collecting urine is comprised of plastic tubing.

7. The urine collecting device and method thereof as set forth in claim 1, wherein said clamping means having a pair of opposing rigid flat tab portions having first and second end portions, said tab portions connected at a first end by a spring, said tab portions second ends being normally distal and urged towards the said opposing tab portion by said spring.

8. The urine collecting device and method thereof as set forth in claim 1, wherein said tube for collecting urine in fluid communication with the hose adaptor, comprises a collapsible tube which is capable of storing a volume of urine and other bodily fluids emanating from a male penis, wherein said volume is at least that of a typical fluid discharge from a person's bladder.

9. The urine collecting device and method thereof as set forth in claim 2, wherein said means for securing the discharge orifice at the adjacent the discharge end comprises an
adjustable strap around the ankle for compressing the tube to close the flow path of liquid.

10. The method of collecting urine comprising the steps of providing a waist harness with means for securing a male penis;
connecting means for securing to a nozzle end;
fluidly coupling said nozzle end to a tubular storage collector;
providing a clamping means adjacent opposite end of said tubular storage means; and
fastening a discharge end of said tubular means to an individual's leg adjacent and above the ankle, thereby concealing said tubular storage means within a man's pant leg so as to avoid visual detection of said tube.
